(19) United States
(12) Patent Application Publication
(10) Pub. No.: US 2009/0104253 A1
(43) Pub. Date: Apr. 23, 2009

(54) Hemostatic biocidal dressing

(76) Inventors: G. Alan Thompson, Ottawa (CA); Richard Eward Brois, SR., Raeford, NC (US); Christopher Matthew Scheuerman, SR., Sanford, NC (US); David Curry Pitts, JR., Spring Lake, NC (US)

Correspondence Address: BERGMAN & SONG, LLP, P.O. BOX 400198, CAMBRIDGE, MA, 02140, US

(21) Appl. No.: 12/156,942

(22) Filed: Jun. 4, 2008

Related U.S. Application Data
(60) Provisional application No. 60/933,027, filed on Jun. 4, 2007.

(51) Int. Cl. A01N 43/16; A61K 9/70; A01N 59/16; A61F 13/15

(52) U.S. Cl. 424/449; 604/367; 514/55; 424/618

(57) ABSTRACT

A hemostatic biocidal dressing includes pro-coagulant and biocidal agents providing a physical barrier to bleeding as well as chemical action to promote blood coagulation and thereby arrest coverage. At the same time, biocidal properties of the dressing prevent the introduction of infected agents and the establishment of infection within wounded tissue. A silverized textile provides biocidal action while a granulated hemostatic agent disposed within interstitial regions between polymer fibers provides coagulating action.

This application claims the benefit under 35 U.S.C. § 119(e) of U.S. Provisional Application No. 60/933,027, entitled “MILITARY HEMOSTATIC BIOCIDAL TRAUMA DRESSING,” filed Jun. 4, 2007, the contents of which are hereby incorporated by reference in their entirety.

### FIELD OF THE INVENTION

The present invention relates to medical consumables and more particularly to wound dressings.

### BACKGROUND

When physical trauma or medical treatment results in a significant breach of the skin, blood loss and infection can pose serious risks to patient survival. It is essential to quickly control hemorrhage and to limit the entry of infective agents, such as microbes, into a wound. The exigencies of combat medicine make achieving these goals particularly important. In combat, wounds are often life-threatening and the time pressures always associated with treating hemorrhage may be amplified by ongoing hostilities.

It has long been known to apply a tourniquet to a significant wound to arrest major blood loss. A major disadvantage of applying a tourniquet is that a substantial amount of undamaged issue may be deprived of circulation and necrotize, broadening corporeal damage beyond the original wound and significantly increasing the risk of gangrene.

More recently, astringents, clotting factors and coagulants have been applied to wounds in an effort to accelerate blood clotting, and thereby arrest hemorrhaging. Typically, a coagulant powder is sprinkled onto a wound and absorbent dressing is applied to the wound with compression. Often initial manual compression with a dressing is followed by binding the absorbent dressing to the wound region with integral and/or separate bandages.

The application of a coagulant powder is limited in its effectiveness by a tendency of rapidly flowing blood to wash the coagulant powder out of the wound, and by a tendency of the powder to blow away from the wound in the presence of rain, wind and air flows generated by the blades of evacuation helicopters. In addition to limiting the delivery of coagulant powder to a wound, thereby rendering it ineffective, wind can blow the coagulant into the eyes and/or respiratory system of a first responder, limiting that person's effectiveness in treating the patient and others, and avoiding injury to him or herself.

### SUMMARY

The present invention provides an integrated system for arresting bleeding, suppressing infection and reducing pain at a traumatic injury site. In surprising contrast to previously available treatments, a dressing according to principles of the present invention can be applied to a wound to rapidly arrest bleeding, form a physical and chemical infection barrier, and stabilize the wound area pending further treatment. In addition, according to various embodiment, the dressing provides an analgesic function, reducing pain related to the wound.

According to one embodiment, the dressing includes a highly absorptive pad that includes both a biocidal component and a coagulation enhancing component. In one surprising aspect of the invention, according to one embodiment, the presence of a metallic coating on an external surface of a fiber of the pad serves both to enhance absorbency of the pad and to provide biocidal functionality. In another surprising aspect of the invention, according to one embodiment, a portion of the pad serves as a filter, whereby blood emerging from a wound under treatment is separated into component parts. Some or all platelets are retained in a first spatial region of the pad, thereby encouraging rapid coagulation, whereas some or all plasma is allowed to pass through the first region of the pad into a second region of the pad. In the second region of the pad, the plasma is brought into contact with a reactive material. According to one embodiment of the invention, the reactive material reacts with the plasma to form a substantially rigid and substantially impermeable barrier to further plasma migration. In a still further surprising aspect of the invention, and one that is remarkable to a practitioner of ordinary skill and creativity, the novel combination of a high surface area fiber, a silver coating, and a coagulant material such as, for example, Chitosin, results in hemostasis substantially faster than previously available, while providing the additional benefit of an integrated biocidal functionality. Thus, in contrast to previous approaches involving using manual or device-assisted compression, along with separately applied coagulation enhancing materials, the device of the present invention provides unexpectedly superior performance.

These and other advantages and features of the invention will be more readily understood in relation to the following detailed description of the invention, which is provided in conjunction with the accompanying drawings.

### BRIEF DESCRIPTION OF THE DRAWINGS

FIG. 1 shows, in cutaway perspective view, a portion of a hemostatic biocidal dressing according to one embodiment of the invention;

FIG. 2 shows a magnified surface portion of a dressing according to one embodiment of the invention;

FIG. 3 shows a magnified surface portion of a dressing according to one embodiment of the invention;

FIG. 4 shows a magnified surface portion of a dressing according to one embodiment of the invention;

FIG. 5 shows a magnified surface portion of a dressing according to one embodiment of the invention;

FIGS. 6A-6G show various exemplary profiles of respective fiber materials according to various embodiments of the invention;

FIGS. 7A-7E show various exemplary profiles of respective fiber materials according to various embodiments of the invention;

FIG. 8 shows, in perspective view, a portion of a fiber material including an interstitial particulate material according to an aspect of one embodiment of the invention;

FIGS. 9A-9F show, in graphical form, various exemplary distribution profiles of active materials within a wound dressing according to respective embodiments of the invention;

FIGS. 10A-10B show various exemplary geometrical configurations of respective wound dressings according to embodiments of the invention;

FIG. 11 shows a portion of an exemplary wound dressing and method according to one aspect of the invention, the dressing and method being adapted for treatment of a substantially convex wound region;

FIG. 12 shows a portion of an exemplary wound dressing and method according to one aspect of the invention, the dressing and method being adapted for treatment of a substantially concave wound region;

FIG. 13 shows a portion of a wound dressing according to one embodiment of the invention;

FIGS. 14A-14C show various exemplary configurations of respective wound dressings according to embodiments of the invention;

FIGS. 15A-15C show various aspects of exemplary wound dressings according to embodiments of the invention;

FIGS. 16A-16B show respective wound dressings including tourniquet portions according to embodiments of the invention;

FIGS. 17A-17C show various aspects of exemplary wound dressings according to embodiments of the invention;

FIGS. 18A-18B show various aspects of exemplary wound dressings according to embodiments of the invention;

FIG. 19 shows a portion of packaged wound dressings according to embodiments of the invention; and

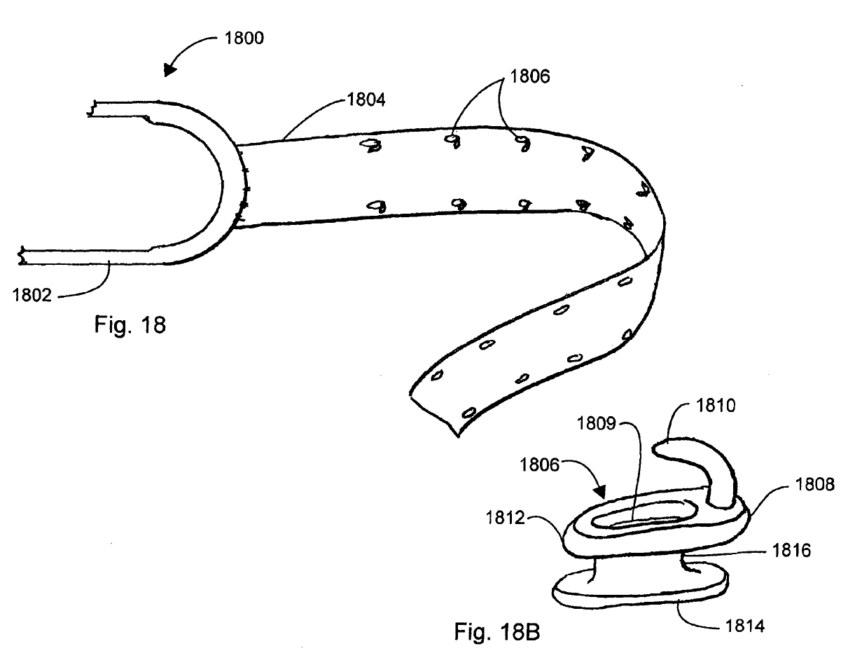

FIGS. 18A-18B show various aspects of exemplary packaged wound dressings according to embodiments of the invention.

### DETAILED DESCRIPTION

The following description is provided to enable any person skilled in the art to make and use the disclosed inventions and sets forth the best modes presently contemplated by the inventors of carrying out their inventions. In the following description, for purposes of explanation, numerous specific details are set forth in order to provide a thorough understanding of the present invention. It will be apparent, however, to one skilled in the art that the present invention may be practiced without these specific details. In other instances, well-known structures and devices are shown in schematic form in order to avoid unnecessarily obscuring the present inventions.

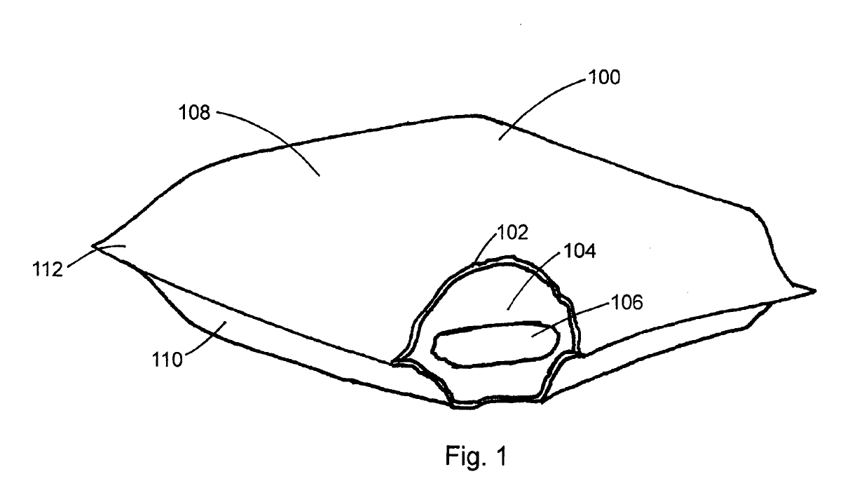

FIG. 1 shows, in cutaway perspective view, a wound dressing 100 according to one embodiment of the invention. The illustrated wound dressing 104 includes an outer layer or envelope 102 including a porous region. Disposed within the envelope 102 is a first transfer layer 104. Disposed within the first transfer layer 104 is a second hemostatically active layer 106. In certain embodiments, the envelope 102 includes first 108 and second 110 substantially flexible sheets coupled to one another at a peripheral bond region 112. In various embodiments of the invention, bond region 112 includes a bonding device such as for example, stitching, an adhesive material, a thermal weld, an ultrasonic weld, a mechanical fastener such as a rivet, a snap, and a zipper, and combinations thereof. In other embodiments of the invention, the envelope 102 is formed as an integral unit.

In various embodiments of the invention, and as will be discussed in additional detail below, one or more of the envelope 102 the first transfer layer 104 and the second hemostatically active layer 106 includes a respective fiber material. In various embodiments of the invention the respective fiber material includes a biocidal material. In various embodiments of the invention, the respective fiber material includes a fiber having a cross-section adapted to provide a high degree of capillary action (a high capillary cross-section). In still further embodiments of the invention, the respective fiber material includes a fiber having a metallic portion or component. In certain embodiments of the invention, a fiber batting is employed with or without hemostatically active material. In other embodiments of the invention a felt material is employed with or without hemostatically active material.

According to still further embodiments of the invention, one or more of the envelope 102, the first transfer layer 104 and the second hemostatically active layer 106 includes a non-fibrous material of appropriate characteristics. In further embodiments of the invention, as will be described below, fewer or more layers of material are present.

Without intending to be bound to a theory of operation, it is noted that the combination of astringent or other hemostatically active materials with bodily fluids can result in adverse consequences to adjacent tissue and, in some circumstances, produce sufficient energy to damage adjacent tissue or structures. For example, an exothermic reaction can, under some circumstances, produce sufficient energy to damage adjacent tissue.

Out of careful study, thoughtful effort and creativity, the inventors have come to understand that an intervening layer of absorbent material can transfer a fluid including water, such as, for example, blood or plasma, to a hemostatic agent material, where the hemostatic agent material is disposed comparatively distal with respect to the wound and surrounding tissue. Accordingly, the activity of the hemostatic agent as it comes into contact with the transferred fluid is non-damaging to the wound and surrounding tissue. In certain embodiments of the invention, it is believed that silverized fibers, and particularly high-surface-area silverized fibers, are exceptionally effective in transferring plasma from a wound to a distally disposed hemostatic agent material. According to certain embodiments, silverized fibers are of particular value in selectively transferring plasma while retaining blood cells in a region distal to the hemostatic agent material.

Surprisingly, the application of a metallic coating to high surface area/hi capillary action material serves to strongly increase capillary action and enhance the transfer of fluid between a wound and a hemostatic material arranged in a dressing as described above. Also surprisingly, a metallic coating including metallic silver has the ability to release ions into adjacent fluids and consequently provide a biocidal and analgesic effect in relation to a wound, while simultaneously promoting capillary transfer of fluids to a hemostatic material such as a coagulant. Thus, the novel invention disclosed herewith exhibits it a surprising and remarkable synergy resulting from the combination of hemostatic material with a silverized transfer material to render a wound dressing effective to provide rapid and non-tissue-damaging hemostasis while concurrently providing beneficial biocidal and analgesic effects.

More generally, it should be understood that a transfer material can be used to transfer plasma away from a wound to, for example, a hemostatic material while simultaneously transferring a pharmaceutical material toward the wound. The pharmaceutical material can include, for example, an analgesic, an antibiotic, a sedative, a vaccine, a genetic transfer vector, a nanoscale material, a nutrient, a vitamin, or any other material determined appropriate for the treatment or well-being of the patient.

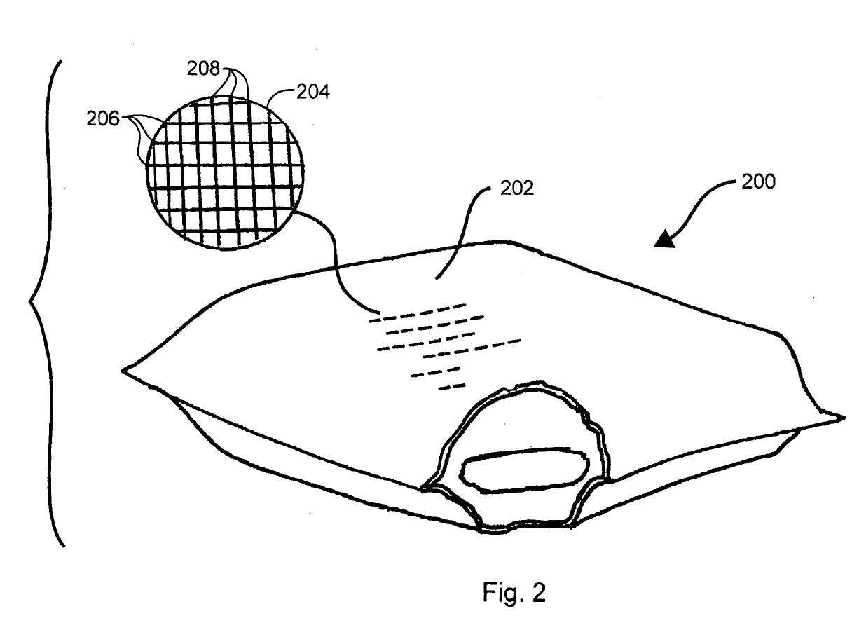

With the foregoing in mind, and in light of the exemplary embodiment illustrated in FIG. 1, one of ordinary skill in the art would understand that a wide variety of materials are properly employed as outer porous layers in respective embodiments of the invention. Thus, as shown in FIG. 2, one exemplary embodiment of the invention includes a wound dressing 200 with an envelope 202 including a material of woven fibers. As shown in a magnified view 204, according to one embodiment, the woven fibers include a first plurality of warp fibers 206 and a second plurality of wove fibers 208 with the warp fibers and wove fibers arranged in substantially perpendicular relationship with respect to one another. As will be discussed in additional detail below, these fibers include, in certain embodiments, high surface area capillary fibers and fibers having a surface treatment such as a metallic coating and such as a coating including silver. In certain embodiments, the fibers include homogenous fibers or an organized or randomized mixture of inhomogenous fibers. In various embodiments, the envelope includes at least one region that is substantially porous and adapted to transfer a fluid such as, for example, plasma, from a first side to a second side thereof.

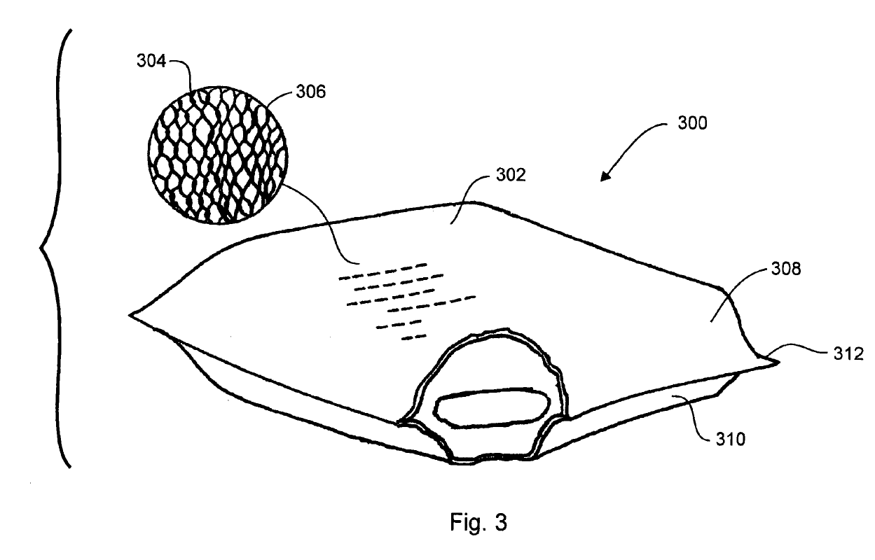

FIG. 3 shows a further embodiment of a wound dressing 300 according to the invention. As shown, the wound dressing includes an envelope 302 having a substantially porous region including knitted fibers. The knitted fibers 304 are shown in magnified view 306. As in the embodiments discussed above, in various embodiments, the knitted fibers 304 include high capillary action fibers. In certain embodiments the knitted fibers 304 include metallic material such as filler. In other embodiments, the knitted fibers 304 include a metallic coating or other metallic surface region. According to certain embodiments, the envelope 302 includes a single integral knitted tube. In other embodiments a knitted portion 308 is coupled to a non-knitted portion 310 at a boundary region 312. In certain embodiments, the non-knitted portion 310 includes a woven portion. In other embodiments, the non-knitted portion 310 includes a sheet material such as, for example, a sheet polymer material. According to various embodiments of the invention, different surface regions of the envelope 302 have respective surface characteristics such that a particular surface region is adapted for application to a wound, or such that different surface regions are adapted for application to respective different classes or types of wound.

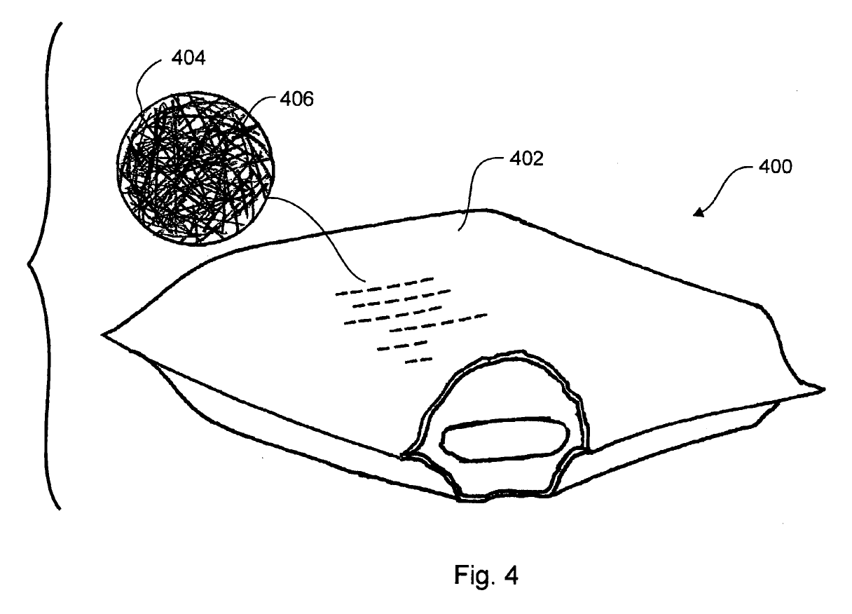

FIG. 4 shows a still further embodiment of the invention including a wound dressing 400 having an envelope 402 including a non-woven fibrous textile material. As shown in a magnified view 404, certain embodiments of the invention include a non-woven textile having a plurality of substantially randomly oriented fibers 406. In other embodiments of the invention, as would be understood by one of skill in the art, fibers of the non-woven textile are distributed in various non-random orientations including, for example, periodic and a periodic perpendicular and/or radial orientations. In certain embodiments, this non-woven fibrous textile material is a felted material. In certain embodiments, the fibers include a metallic material such as, for example, a metallic silver coating.

In certain embodiments of the invention, the metallic silver coating includes a substantially pure silver coating. In certain embodiments of the invention the silver coating includes a silver material having a purity of at least about 99.99% pure. In another embodiment of the invention, the silver coating includes a silver material having a purity of the least about 99.995% pure. In another embodiment of the invention, the silver coating includes a silver material having a purity of the least about 99.9995% pure. In one embodiment of the invention, the metallic silver coating has a thickness of at least about 50μ. In another embodiment of the invention, the metallic silver coating has a thickness of between about 1μ and about 50μ.

According to various embodiments of the invention, an integrity of the non-woven textile is maintained by the formation of bond interfaces between various fibers of the non-woven textile material. According to various embodiments of the invention, the bond interfaces include, for example, chemical adhesive bond interfaces, thermally welded bond interfaces resulting from the application of, for example heat and/or ultrasonic energy, bond interfaces formed by metallic welds between the above-noted metallic materials by, for example the deposition of further medal in an electrochemical process or the application of heat and/or pressure and/or ultrasonic and/or electrical energy.

In the various embodiments, as discussed above, the envelope includes fibrous material formed of fibers including a polymeric material. In certain embodiments, the polymeric material includes a polyamide material. In exemplary embodiments, the fibers include drawn and undrawn fibers, on grooves, grooved and deep grooved fibers, of natural and synthetic polymer material including, for example, polyaramid, polyethylene, polyester, polyethylene terephthalate, polyhexamethylene adipamide, or polypropylene, wool, cotton, flax and cellulose, and including addition polymers, condensation polymers, thermoplastics and thermoset materials, among others.

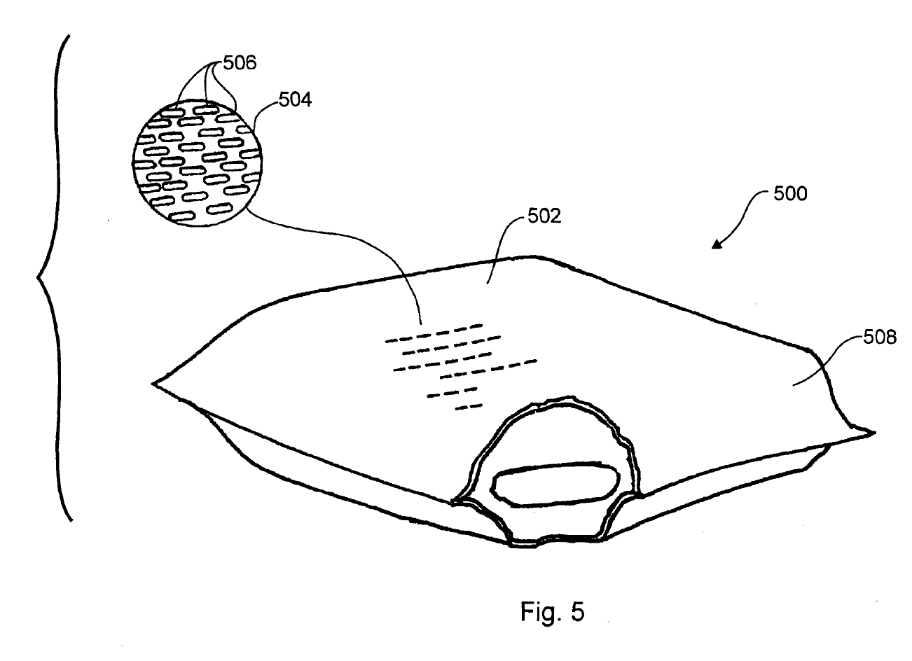

FIG. 5 shows another embodiment of the invention including a wound dressing 500 having an envelope 502. The envelope 502 includes a porous region (as shown magnified 504) with a plurality of holes or pores 506 therethrough. In certain embodiments, the pores are distributed substantially evenly over a surface 508 of the envelope 502. In other embodiments, the pores are concentrated in a particular region of the envelope. In still other embodiments of the invention, the pores include pores of uniform or variegated perimeter shape, size, distribution and density. For example, in certain embodiments, the pores include pores with perimeters that are substantially circular, elliptical, oval, polygonal including triangular, rectangular, pentagonal, hexagonal heptagonal, and/or octagonal. As a creative practitioner of ordinary skill in the art would understand, other perimeter arrangements, including combinations thereof, would fall within the scope of the invention. In various embodiments, the envelope includes a material such as a reinforced or unreinforced polymer material. In certain embodiments this material is a natural polymer or a synthetic polymer. In certain embodiments, the polymer material includes one or more of a polymer and an elastomer such as, for example, polyethylene, polyethylene terephthalate (polyester) including, for example, bi-axially oriented polyethylene terephthalate (Mylar®), polyamide (Nylon®), polypropylene, polyaramid, polyurethane, neoprene, butadiene, Polyetheretherketone (PEEK), ceramic material and other appropriate materials such as are known or may become known in the art. According to various embodiments of the invention, the polymer material includes a textile material and/or fiber reinforcement material.

Referring again to FIG. 1, certain embodiments of the invention include a transfer layer 104 and a hemostatically active layer 106 disposed within a cavity defined by an inner surface of the envelope 102. According to certain embodiments of the invention, the transfer layer 104 includes a bat of fibrous material. According to certain embodiments of the invention, the fibrous material includes a polymer fiber material having a high capillary action fiber with a relatively large silverized surface area adapted to promote high capillary transfer of fluids from the envelope 102 to the hemostatically active material 106. In various embodiments, the fiber material includes a homogenous fiber material. In other embodiments, the fiber material includes a mix of fiber materials including one or more of un-grooved, grooved and deep grooved fiber materials, either uniformly oriented or randomly oriented. Also in various embodiments, the fibers include one or more of heavy denier, fine denier and microfibers, alone or in accommodation. In addition, in various embodiments, the fibers include short staple, long staple and continuous filament/TOW fibers, alone or in combination.

In certain embodiments of the invention, a silverized fiber material included in the wound dressing includes a silverized fiber material prepared according to the disclosure presented in one or more of U.S. Pat. No. 7,172,785 entitled Process for Deposition of a Metal on a Surface and U.S. Pat. No. 6,726,964 entitled Ultrasonic Process for Autocatalytic Deposition of a Metal on a Micro-Particulate.

In other embodiments of the invention, transfer layer 104 includes another material adapted to promote a transfer of fluid between envelope 102 and the hemostatically active material 106. Thus, in certain embodiments, transfer material 104 includes a more or less porous, more or less flexible material such as an open cell polymer foam material (including, for example, a natural or synthetic sponge material), a sintered or un-sintered particulate material, and aerogel material, a cellulosic material including, without limitation, a felt, a textile, a leather, an aerogel, a paper material or other more or less porous material as known in the art.

In embodiments including a porous material the pores may be of, for example, circular shape, elliptical shape, oval shape, polygonal shape including triangular, rectangular, pentagonal, hexagonal, heptagonal, octagonal, etc. In other embodiments, the pores may be of random shape and/or size and of substantially uniform or mixed size.

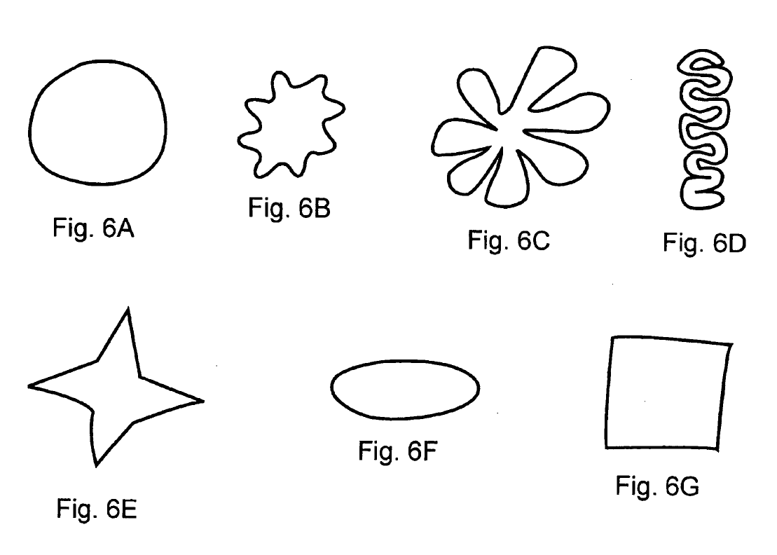

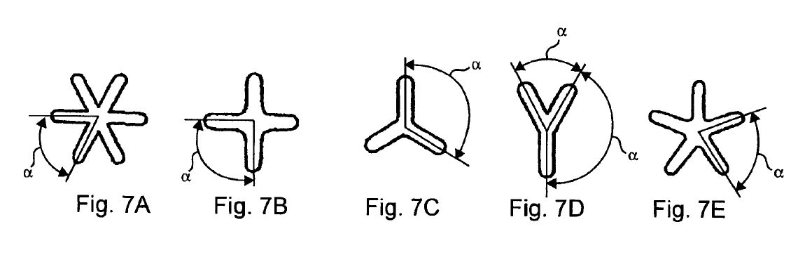

FIGS. 6A-6E shows, in cross-sectional view, various exemplary profiles of fibers including high-surface area fibers used in various respective embodiments of the invention. FIG. 6A shows an exemplary fiber of substantially circular cross-section. FIG. 6B shows an exemplary fiber of generally circular fluted cross-section. FIG. 6C shows an exemplary fiber of generally circular deeply grooved cross-section. FIG. 6D shows an exemplary fiber of generally serpentine cross-section. FIG. 6E shows an exemplary fiber of generally stellate cross-section. FIG. 6F shows an exemplary fiber of generally elliptical cross section, and FIG. 6G shows an exemplary fiber of generally polygonal cross-section. The creative practitioner of ordinary skill in the art will appreciate a wide variety of other fiber profiles, including mixtures of profiles, are possible. Further exemplary profiles are illustrated and discussed in U.S. Pat. No. 6,610,903 entitled Materials for Fluid Management in Personal Care Products, FIG. 3 of which is reproduced herewith as FIGS. 7A-7E.

In various embodiments, the fibers include one or more of cotton, flax, hemp, polyamide, polyethylene (including low-density linear polyethylene, high-density polyethylene, ultrahigh molecular weight polyethylene), polyethylene terephthalate (polyester), copolyester, polypropylene, poly lactic acid, poly 1, 4 cyclohexane dimethyl, terephthalate (PCT), and glycol-modified PET (PETG), among others.

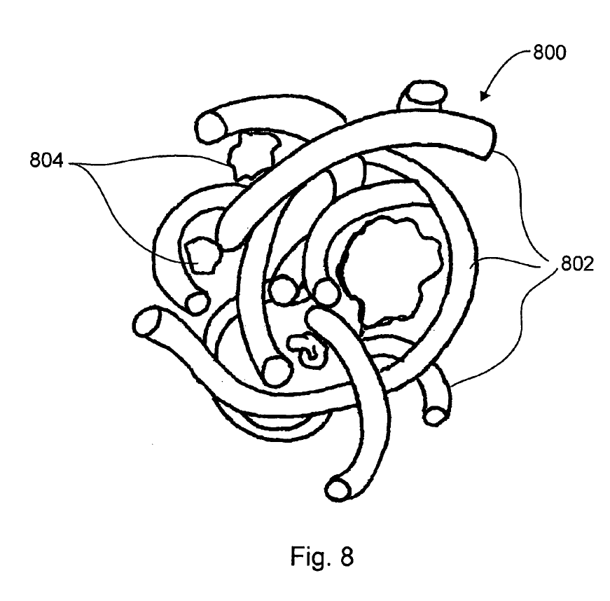

As discussed above in relation to FIG. 1, the hemostatically active layer 106 of the wound dressing 100 is disposed inwardly of transfer layer 104. In one exemplary embodiment, this hemostatically active layer includes a substantially fibrous material and a hemostatically active particulate material disposed interstitially between the fibers of the fibrous material. In other embodiments, the hemostatically active material is disposed in a coating on the transfer material: for example as a pharmaceutical coating on a deep grooved fiber. In still another embodiment of the invention, the hemostatically active material includes a liquid. In still another embodiment of the invention, the hemostatically active material includes a gel. In yet another embodiment of the invention, the hemostatically active material includes a material encapsulated within a second material, such as a gel or powder disposed within a frangible capsule of, for example, gelatine or a polymer material. In yet another embodiment of the invention, the fibers are intrinsically hemostatic and in still another embodiment of the invention, sheets, strips, disks, spheres, or any other appropriate arrangement of hemostatically active material is employed. Accordingly, FIG. 8 shows, in perspective cutaway view, a portion of an exemplary hemostatically active region 800 according to one aspect of the invention. As shown, the hemostatically active region 800 includes a plurality of fibers 802 and a further plurality of particles 804 of hemostatically active material disposed interstitially between the fibers. In certain embodiments, the hemostatically active material particles 804 includes substantially non-porous particles. In other embodiments, the hemostatically active material particles include substantially porous particles. In certain embodiments, the particles include faceted, smooth or corrugated surfaces respectively.

According to certain embodiment of the invention, the fibers include fibers having high capillary action and high surface area fibers. Likewise, in various embodiments of the invention, the fibers include silverized fibers or otherwise metallically coated, embedded or otherwise activated fibers. In some embodiments of the invention, the fibers of the hemostatically active region are substantially identical to fibers of a transfer region. In certain embodiments of the invention, the silverized fibers include fibers having silver ironically bonded to nylon. In certain embodiments of the invention, the silver is ironically bonded within deep channels of a deep grooved nylon fiber material.

In certain embodiments of the invention, a hemostatically active material is substantially uniformly distributed within a hemostatically active layer. In other embodiments of the invention, a hemostatically active material is disposed in a nonuniform distribution within the in the hemostatically active layer. For example, in certain embodiments of the invention the density distribution of hemostatically active material increases monotonically with distance from an edge of the hemostatically active region. In other embodiments of the invention, a distribution of the hemostatically active material varies cyclically and in still other embodiments the density of hemostatically active material within the hemostatically active region varies stochastically as a function of location over the region.

In certain embodiments of the invention, hemostatically active material is distributed throughout the transfer region 104 within the envelope 102 of FIG. 1. According to certain embodiment of the invention, the distribution of the hemostatically active material varies substantially monotonically from a relatively low density distribution of hemostatically active material disposed relatively proximal to an internal surface of the envelope 102 to a relatively high density distribution of hemostatically active material disposed relatively distal to the internal surface of the envelope.

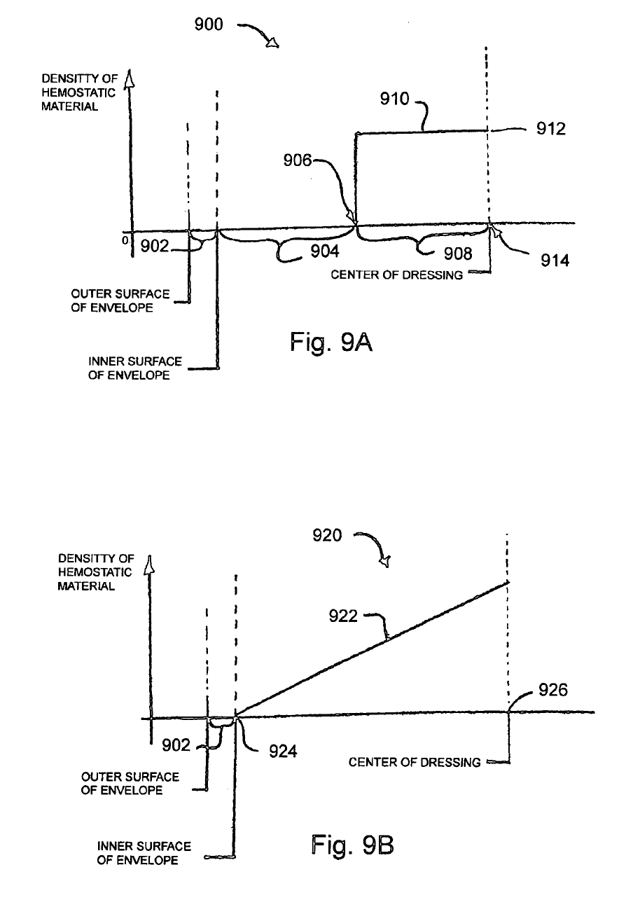

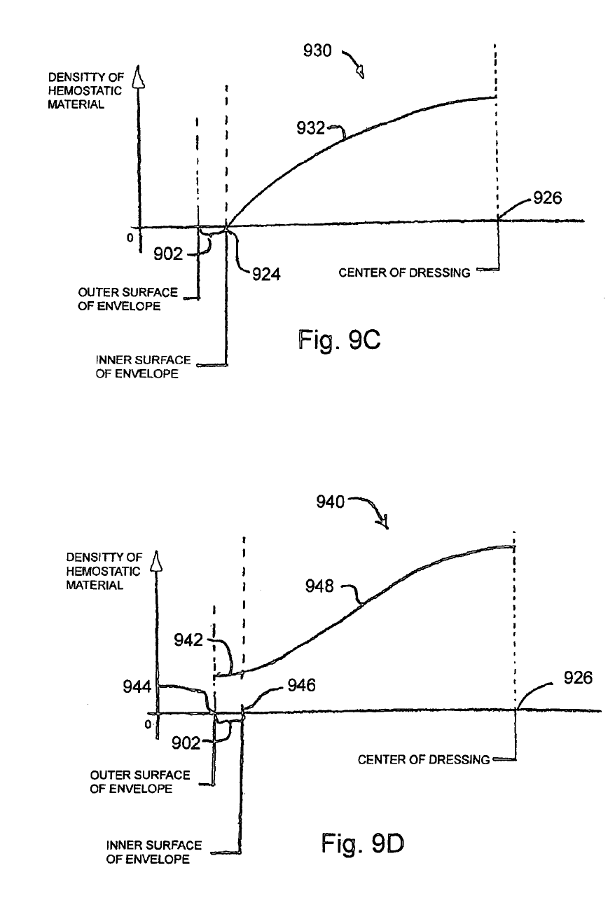

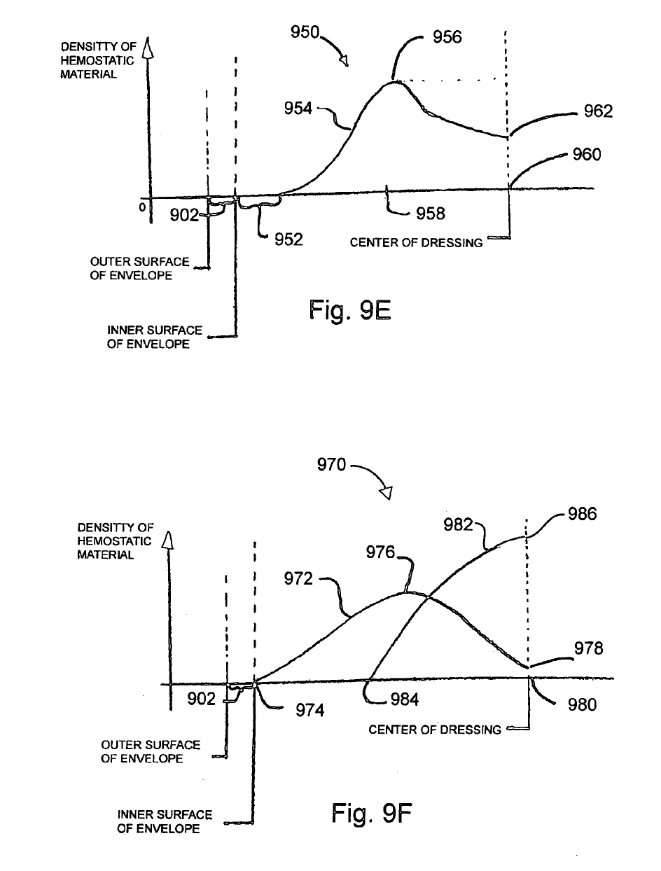

Accordingly, FIGS. 9A-9F show, in graphical form, various exemplary distributions of hemostatically active material within and throughout respective exemplary dressings. As indicated, a spatial concentration of hemostatically active material is plotted with respect to spatial distance across the device. The graphs of FIGS. 9A-9F assume a spatially symmetrically configured dressing and distribution of hemostatically active material. The creative practitioner of ordinary skill in the art would appreciate, however, that a wide variety of arrangements are possible, and appropriate to various applications and general use or special use dressings, and that the illustrated distributions are merely exemplary of many alternative embodiments.

FIG. 9A shows a first distribution 900 of hemostatically active material. According to the embodiment of FIG. 9A the envelope 902 is substantially free of hemostatically active material, as is a first transfer layer 904. At an interface 906 between the transfer layer and a second hemostatically active layer 908 the concentration 910 of hemostatically active material rises precipitously, and in some embodiments substantially instantaneously, from zero to a specified level 912. In the illustrated embodiment, the concentration/distribution of hemostatically active material 910 is substantially constant across the hemostatically active layer 908 between the interface 906 and the center of the dressing 914. Thus, in a substantially symmetrical dressing, the concentration of hemostatically active material is substantially constant throughout the hemostatically active layer 908.

FIG. 9B shows a distribution of hemostatically active material in a wound dressing 920 according to an alternative embodiment of the invention. In dressing 920, envelope 902 is substantially free of hemostatically active material. Within the dressing, a concentration 922 of hemostatically active material rises substantially linearly from an inner surface 924 of the envelope 902 to a center 926 of the dressing.

FIG. 9C shows a distribution of hemostatically active material in a wound dressing 930 according to another embodiment of the invention. In dressing 930, envelope 902 is substantially free of hemostatically active material. Within the dressing, a concentration 932 of hemostatically active material rises nonlinearly and, in the illustrated embodiment, substantially monotonically from an inner surface 924 of the envelope 902 to a center 926 of the dressing. In the illustrated embodiment, the concentration 932 of hemostatically active material is a substantially parabolic function of distance taken with respect to interface 924.

FIG. 9D shows a distribution of hemostatically active material in a wound dressing 940 according to still another embodiment of the invention. In dressing 940, envelope 902 includes a relatively low, substantially constant concentration 942 of hemostatically active material is present throughout the envelope 902 between an external surface 944 and an internal surface 946 of the envelope. Within the dressing, a concentration 922 of hemostatically active material rises, here nonlinearly, from the inner surface 946 of the envelope 902 to the center 926 of the dressing.

FIG. 9E shows a distribution of hemostatically active material in a wound dressing 950 according to yet another embodiment of the invention. In dressing 950, the envelope 902 is substantially free of hemostatically active material, as is a first region 952 of the dressing 950 disposed inwardly of the envelope 902. Further inward within the dressing 950, a concentration 954 of hemostatically active material, rises to a peak concentration 956. The peak concentration 956 occurs at a region 958 disposed outwardly of a center 960 of the dressing 950. In a further region of the dressing 962 disposed between region 958 and center 960, the concentration 954 of the hemostatically active material decreases to a local minimum 962. In certain embodiments of the invention, local minimum 962 corresponds to a concentration of substantially zero. In certain embodiments of the invention a concentration of substantially zero is achieved outwardly of the center 960 of the dressing.

FIG. 9F shows a distribution of hemostatically active material in a wound dressing 970 according to still a further embodiment of the invention. In dressing 970, the envelope 902 is substantially free of hemostatically active material. A first concentration 972 of a first hemostatically active material rises from a relatively low-level, or zero, adjacent an inner surface 974 of envelope 902 to a peak value 976 and a region of the dressing 970 between inner surface 974 and a center of the dressing 980. Thereafter, concentration 972 decreases to a sub-peak value 978 at a region, here shown as adjacent center 980, of the dressing 970. A concentration 982 of a further material is shown to increase from a further region 984 of the dressing 972 of value, here shown as a maximum value 986 in proximity to the center 980 of the dressing. According to various modes of the invention, the second concentration 982 is a concentration of a second hemostatically active material. In other embodiments of the invention, the second concentration 982 is a concentration of a material adapted to cooperate with, or otherwise enhance, an activity of the hemostatically active material of concentration 972. In certain embodiments of the invention, the material of concentration 982 is adapted to react endothermically with a material such as, for example, blood plasma.

In light of the disclosure provided above, and particularly in relation to FIGS. 9A-9F, the creative practitioner of ordinary skill in the art would appreciate a wide variety of distributions of physical and chemical constituents, including but not limited to hemostatically active materials, are provided in various embodiment of the invention and fall within the scope of the present disclosure.

In certain embodiments of the invention, the hemostatically active material includes particulate material of substantially uniform size. In other embodiments of the invention, the hemostatically active material includes particulate material including particles having respective average diameters marrying over a distribution of sizes. In certain embodiments, the particles of hemostatically active material are have a substantially smooth peripheral surface such as, for example, a substantially smooth substantially spherical surface or a substantially smooth substantially ellipsoidal surface. In other embodiments, the particles of hemostatically active material are rough, groove, pitted, or otherwise have substantially non-smooth peripheral surfaces.

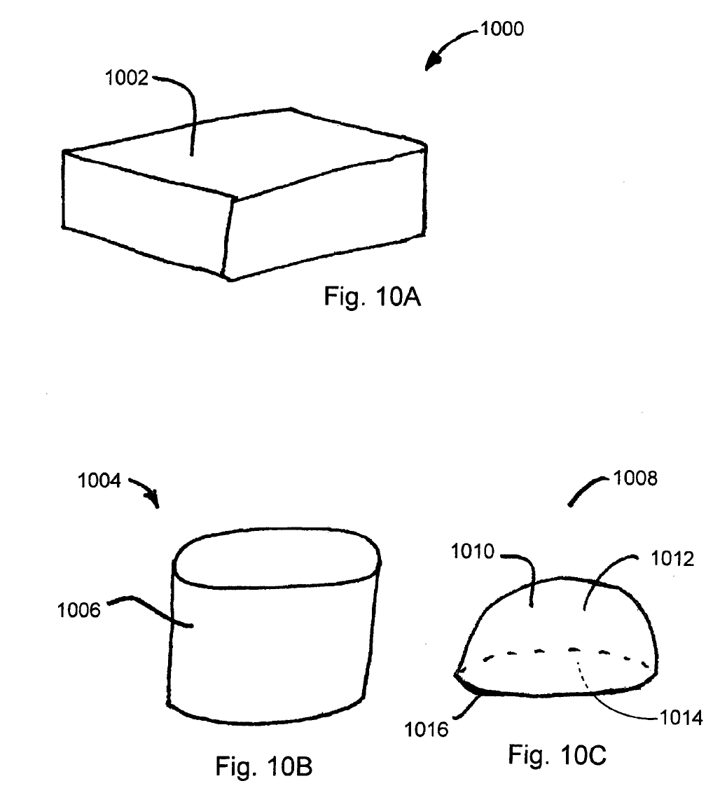

In light of the disclosure above, the creative practitioner of ordinary skill in the art will appreciate that a wide variety of general purpose and special-purpose dressings can be prepared according to the principles of the present invention. Thus, FIG. 10A shows an exemplary dressing 1000 with an external surface 1002 substantially defining a rectangular solid. FIG. 10B shows a further exemplary dressing 1004 with an external surface 1006 substantially defining a cylindrical solid. FIG. 10C shows yet another exemplary dressing 1008 having a surface 1010 including a first substantially hemispherical region 1012 and a second substantially flat region interfacing with region 1012 at a substantially circular boundary 1016.

One of skill in the art will appreciate that the various symmetries and asymmetries presented in the exemplary embodiments illustrated permit the wound dressing to be adapted to various applications. Thus, surface region 1014 of dressing 1008 is readily adapted to be disposed adjacent to a substantially flat wound such as a burn, whereas, under other circumstances, surface region 1012 can be disposed adjacent to an inner surface of a wound cavity. It should be noted that, in many embodiments, the dressing is substantially elastically compressible and thus can conform to a wound of a particular geometry.

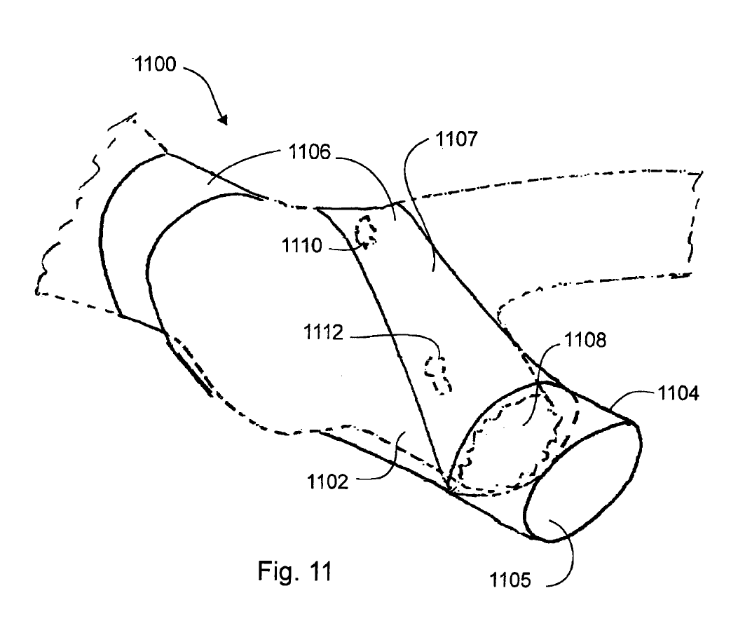

In addition to dressings of various general shapes, certain embodiments of the invention are well adapted for the treatment of particular classes of wounds or for use in particular situations. For example FIG. 11 shows, in perspective view, an exemplary embodiment of a wound dressing 1100 adapted for application to a truncated limb 1102 according to one aspect of the invention. The truncated limb dressing 1100 includes a first enclosing portion 1104 and a second harness portion 1106 coupled to the enclosing portion. The enclosing portion 1104 is adapted to cover and protect the wound, here at a truncated end 1108 of the limb 1102. The harness portion is adapted to be disposed about an adjacent region of a body of the patient, and to locate and support the enclosing portion in substantially fixed location with respect to the truncated end 1108.

In certain embodiments the enclosing portion 1104 includes and outer surface 1105 and a substantially concave surface region defining a cavity within the enclosing portion 1104 adapted to receive the end 1108 of the truncated limb 1102 therewithin. In various embodiments, and in light of the disclosure provided above, the enclosing portion 1104 includes a silver coated fibrous material and a hemostatically active material such as Chitosin. In certain embodiments, the harness portion 1106 includes a substantially elastic material. In other embodiments, the harness portion 1106 includes a substantially inelastic material. In various embodiments, harness portion 1106 is adapted to be disposed in tension so as to apply a force (or pressure) between a wound-contacting surface of enclosing portion 1104 and an end 1108 of the truncated limb 1102.

According to certain embodiments, the harness portion is adapted to cover and protect secondary wounds (e.g., 1110, 1112). Thus in certain embodiment, the harness portion includes one or more of a silver coated fibrous material and a hemostatically active material such as Chitosin. In certain embodiments, harness portion 1106 includes a substantially tubular envelope formed of a substantially elastic material such as a knitted silverized high capillary action fiber material. In certain embodiments of the invention, the dressing 1100 includes a first relatively more elastic inner surface and a second relatively more robust outer surface. One of skill in the art will appreciate a wide variety of inner and outer surface materials would fall within the scope of the invention, including woven, knitted, and nonwoven textile materials, sheet polymer materials, cellulosic materials and other materials known in the art. In one embodiment of the invention, at least one layer of silverized batting 1107, including a hemostatically active particulate material, is disposed within the harness portion 1106 of the dressing 1100. In other embodiments of the invention, a layer of silverized felt material including a hemostatically active particular material is disposed within the harness portion. In another embodiment of the invention, a layer of silverized batting material and/or silverized felt material including a coating of hemostatically active material is included in the harness portion.

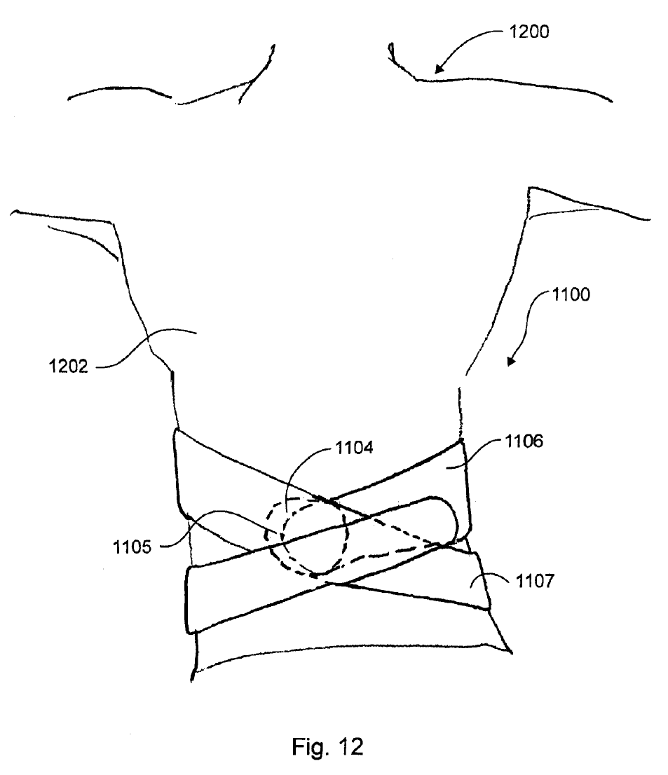

FIG. 12 shows an alternative application 1200 of dressing 1100. In the illustrated application, the dressing 1100 is used to dress a substantially concave wound such as a belly wound. As shown, the enclosing portion 1104 is disposed substantially entirely within the cavity of the wound. In the illustrated application, what was previously disposed as an outer surface 1105 is now disposed substantially in contact with the wounded tissue. The harness portion 1106 is disposed circumferentially about an abdomen 1202 of a patient. The versatility of the invention in its various embodiments utility and is thus illustrated.

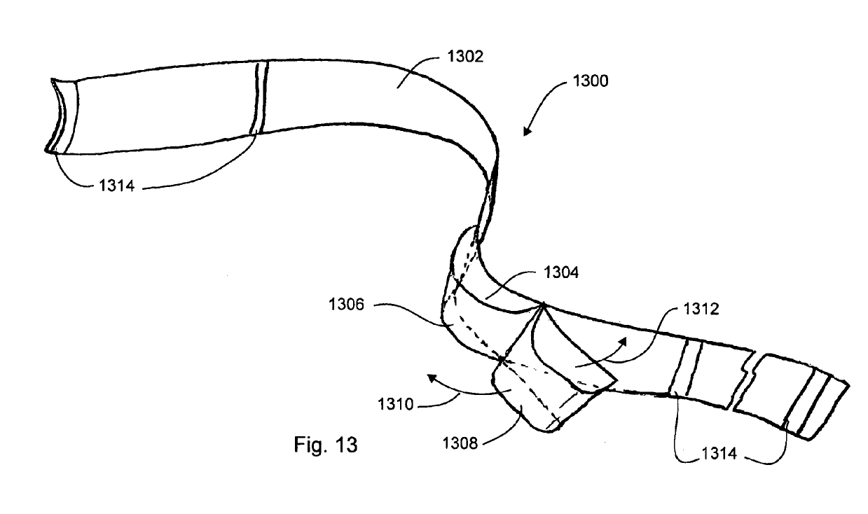

FIG. 13 shows, in perspective view, a further embodiment of the invention including a wound dressing 1300 incorporating various features as described above. The wound dressing 3000 includes a harness portion 1302 and an enclosing portion 1304. In certain embodiments, the harness portion 1302 includes a silverized substantially elastic material; for example a silverized knitted deep groove nylon fiber material. In further embodiments, the harness portion 1302 includes one, two or more layers of silverized material with one, two or more layers of Chitosin impregnated batting.

As illustrated, the enclosing portion includes a first wound covering portion 1306 and a second wound covering portion 1308. In the illustrated embodiment, both wound covering portion 1306 and wound covering portion 1308 are coupled to the harness portion 1302. Wound covering portion 1308 is adapted to be either folded over on top of wound covering portion 1306 1310 so as to provide a relatively thick configuration of enclosing portion 1304. Alternately wound covering portion 1308 is adapted to be unfolded 1312 so as to provide a relatively thin configuration of enclosing portion 1304 but with a larger effective wound covering surface area. One of skill in the art will appreciate that the various hemostatic, biocidal and analgesic features discussed above are readily incorporated into the various embodiments of the invention represented by exemplary wound dressing 1300. In addition, in certain embodiments, the wound dressing of the invention includes to capture mechanism such as, for example, a hook and loop fastener material adapted to hold the field dressing 1300 in one or both of a stored configuration and a deployed configuration.

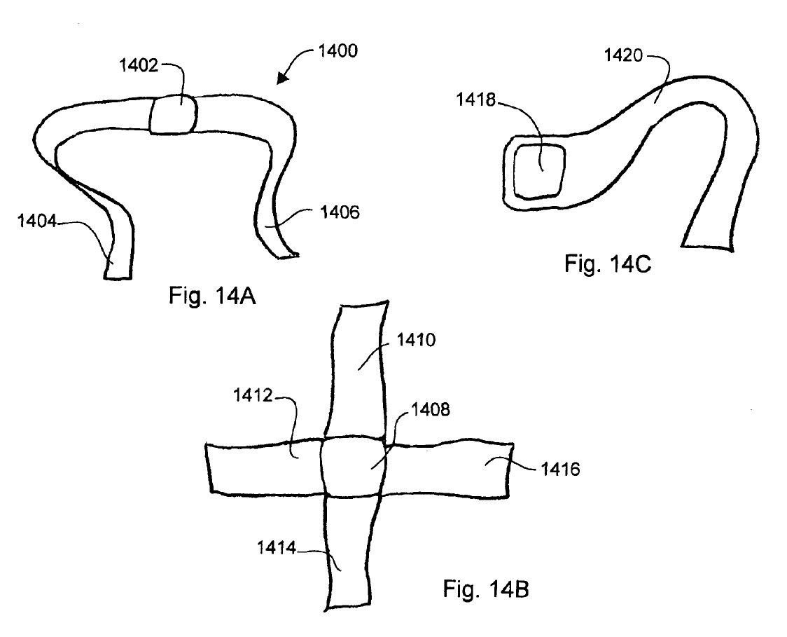

Of course it should be understood that the dressings exemplified by the illustrated embodiments can be prepared in a wide variety of sizes and shapes according to the demands of particular applications and circumstances. Thus for example, FIG. 14A shows a wound dressing 1400 including an enclosing portion 1402 coupled substantially symmetrically between first 1404 and second 1406 harness portions. FIG. 14B shows an enclosing portion 1408 coupled substantially symmetrically between first 1410, second 1412, third 1414, and fourth 1416 harness portions. FIG. 14C shows an enclosing portion 1418 coupled asymmetrically at one end of a harness portion 1420.

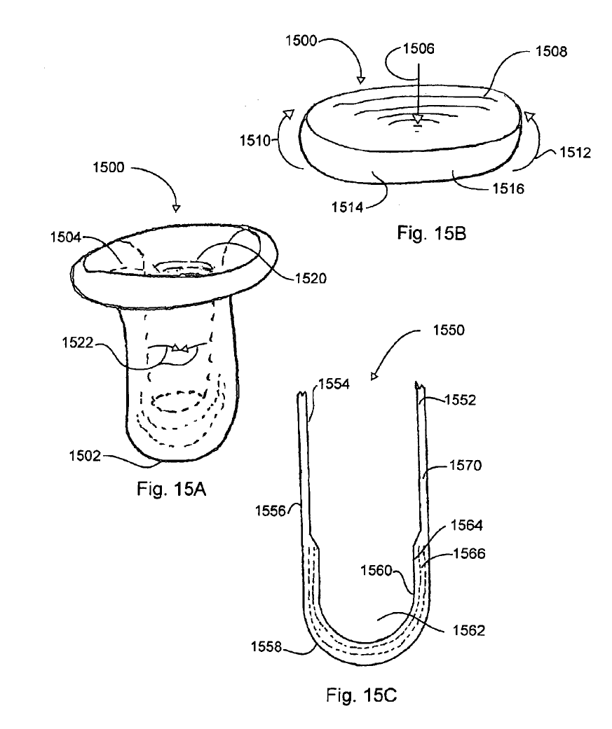

FIG. 15A shows, in perspective view, a wound dressing 1500 according to a further embodiment of the invention. Wound dressing 1500 includes a substantially tubular dressing having a first closed end 1502 and a second open end 1504. As shown in FIG. 15B would dressing 1500 can be disposed, as for storage, in a rolled configuration. Prior to, or in the course of, application a first for us 1506 is applied at a central region of an internal surface 1508 concurrently, curling forces 1510, 1512 are applied at respective outer edge regions 1514, 1516 of the dressing. Consequently, the dressing 1500 tends to unroll and assume the configuration shown in FIG. 15A.

Referring again to FIG. 15A, an internal surface 1520 defines a cavity within the dressing. The cavity is adapted to receive therewithin a truncated, burned, or otherwise injured limb, head, or other appropriately sized body part, for example. Because the transition from a rolled configuration as shown in FIG. 15B to an unrolled configuration as shown in FIG. 15A can be made with a wounded extremity in situ, this transition can be used to facilitate application of the dressing. Thus, according to one embodiment, the invention includes a method of applying a dressing including disposing a rolled dressing adjacent to a wounded extremity and unrolling the dressing over the extremity so as to hold, for example, a hemostatic biocidal material generally in proximity to the wound. It should also be noted that because application of the dressing involves and unrolling of the dressing rather than, for example, a helical winding of the dressing, application of the dressing is often possible significantly faster and with less risk of contamination of wound-interfacing surfaces that is possible in other dressings and methods.

In various embodiments, the dressing includes a substantially elastic material such as, for example, a knitted textile material. In certain embodiments, the new textile material includes a silverized knitted nylon material. One of skill in the art will appreciate that as the dressing 1500 is unrolled over an extremity, the substantially elastic material tends to apply a compressive force 1522 radially inwardly on a corresponding external service region of a wounded extremity. This compressive force tends to hold the dressing in place adjacent to the extremity.

FIG. 15C shows, in cross-section, a further aspect of a dressing 1550 according to one embodiment of the invention. The dressing 1550 includes a substantially elastic sleeve portion 1552 having a first inner surface region 1554 and a second outer surface region 1556. In various monitor the invention, the substantially elastic sleeve portion 1552 includes a polymeric material such as, for example, a woven or nonwoven textile, a knitted textile, a felted textile, an elastomeric polymer sheet material, an elastomeric helical spring material, high surface area high capillary action fiber material, metallized fiber material including silverized fiber material, other appropriate materials as known in the art, and combinations thereof. As illustrated, sleeve portion 1552 includes a closed end 1558 having a further substantially concave (as applied) surface region 1560, defining a cavity 1562 and adapted to receive wounded flesh adjacent thereto.

According to various embodiments of the invention, one 1564, two 1566 or more layers of silverized high capillary action fiber batting are disposed in proximity to inner surface region 1560. According to still further embodiments of the invention, one or more of the layers of batting include a hemostatic material such as, for example, a distributed particulate hemostatic material. In certain embodiments of the invention, a transfer layer is disposed between the inner surface 1560 and the hemostatic material so as to prevent hemostatic material from adversely affecting tissue of the wound or other tissue.

In still further embodiments of the invention, silverized fibers are found throughout the dressing so as to provide beneficial biocidal and analgesic functionality to a primary wound and any proximate secondary wounds. According to one embodiment of the invention at least one layer of silverized batting 1570 including Coagulant particulate material is disposed within the substantially elastic sleeve portion 1552 of the dressing 1550.

It should be noted that the embodiments of the invention exemplified as 1500 and 1550 constitute a self-contained dressing and bandage combination. As such they offer decreased application time in a trauma setting as compared with conventional dressing devices and methods. The Coagulant impregnated batting will significantly increase clotting potential over other dressing materials including, for example, cotton. The silverized elasticized tubing provides immediate compression, antimicrobial protection and analgesic effect. According to certain embodiments, significant analgesic effect is available beginning at a time from about 5 minutes to about 20 minutes after application of the dressing. In various embodiments, the dressing of the invention is robust and readily adapted to storage in a vacuum package for significant shelf life.

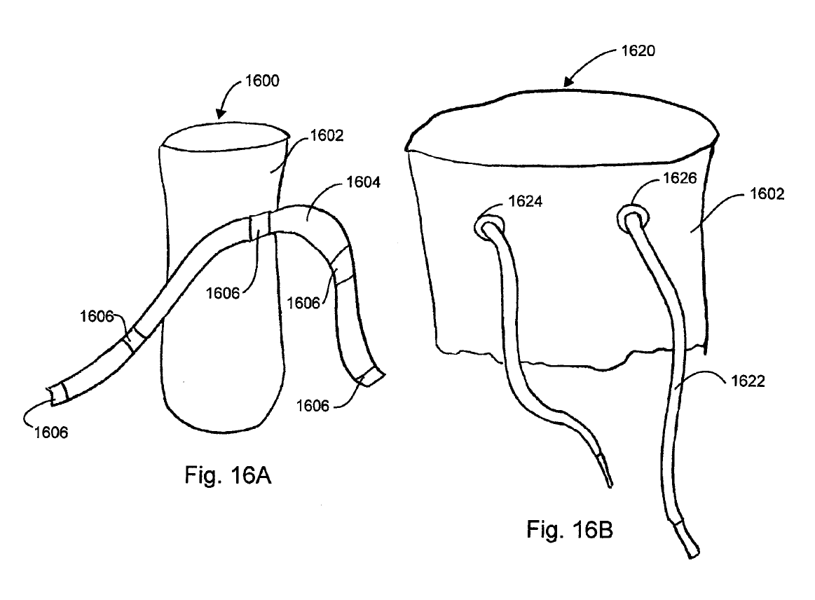

FIG. 16A shows a wound dressing 1600 according to another embodiment of the invention including a substantially tubular portion 1602 and an integrated tourniquet portion 1604. In certain embodiments of the invention, the tourniquet portion 1604 is substantially permanently coupled to the tubular portion. In other embodiments of the invention, the tourniquet portion 1604 is remotely coupled to the tubular portion 1602 by a coupling device 1606 such as, for example, a hook and loop fastener system (e.g. Velcro®). In certain embodiments, the coupling device 1606 is further adaptable for fixing the tourniquet in place to apply tourniquet pressure.

According to still another embodiment 1620 of the invention, as illustrated in FIG. 16B, a tourniquet portion includes a flexible member 1622 such as, for example, a cord or lace. In certain embodiments, the cord or lace is disposed between inner and outer fabric layers of the tubular portion 1602. In certain embodiments of the invention, the likable member passes into and out of an outer fabric layer of the tubular portion through respective first 1624 and second 1626 eyelets.

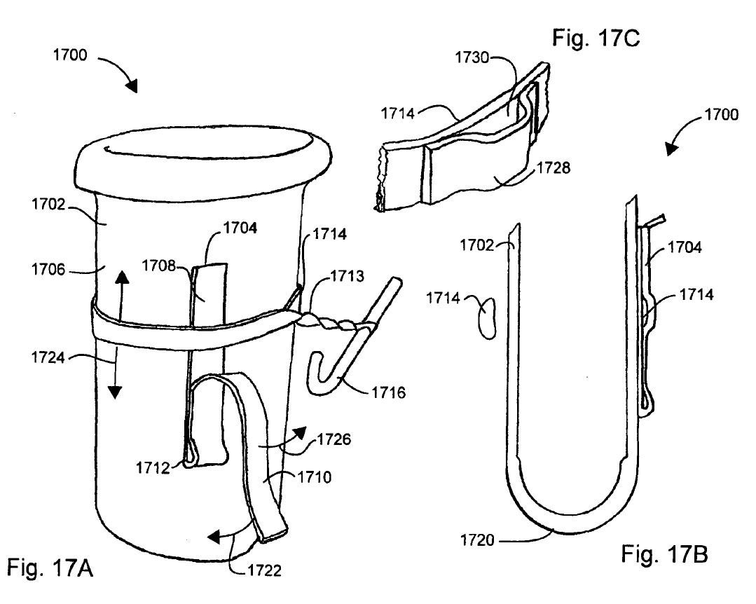

FIG. 17A shows, in perspective view, a further embodiment of the invention including a wound dressing and tourniquet combination 1700. In the illustrated embodiment, the combination includes a dressing portion 1702 adapted to be unrolled over a wound such as a truncated extremity wound. In the illustrated embodiment, a fastening device 1704 is coupled to an external surface 1706 of the dressing portion 1702. In certain embodiments, the fastening device 1704 is a substantially flexible fastening device such as, for example, a hook and loop fastener device. Hook and loop fastener devices are well known in the art, for example under the trade name Velcro®. In the illustrated embodiment, the fastener device 1704 includes a hook portion 1708 and a loop portion 1710. As shown, in one embodiment be hook 1708 and loop 1710 portions are coupled to one another at coupling region 1712. As illustrated, the hook portion 1708 of the fastening device is coupled to external surface 1706 of the dressing portion. One of skill in the art will appreciate that an alternative arrangement, in which the loop portion 1710 of the fastening device 1704 is disposed adjacent to surface 1706, is appropriately employed in certain embodiments.

In the illustrated embodiment, a tourniquet portion 1714 is included with the dressing portion 1702. Also included with the dressing portion 1702, in certain embodiments, is a handle portion 1716. In certain embodiments of the invention, the dressing portion 1702, the tourniquet portion 1714, and the handle portion 1716 are mutually enclosed in a common package. According to certain embodiments of the invention, the tourniquet portion 1714 is disposed within a rolled-up region of the dressing portion 1702 prior to application of the dressing portion to a wound. According to certain amount of the invention, the tourniquet portion forms a continuous loop circumferentially around the outer surface of the dressing portion 1702. In other embodiments of the invention, the tourniquet portion 1714 includes a fastening device adapted to allow an effective circumference of the and tourniquet portion, as applied, to be adjusted.

FIG. 17B shows, in cross-section, a further view of wound dressing and tourniquet combination 1700. In use, the tourniquet portion 1714 is disposed circumferentially around the wound dressing portion 1702. A fastener portion 1704 holds the tourniquet portion adjustably in a particular location with respect to a close end 1720 of the dressing portion 1702.

Referring again to FIG. 17A one sees that once the dressing portion 1702 has been applied to, for example, a truncated limb, a loop portion 1710 of the hook and loop fastener 1704 can be withdrawn 1722 from the hook portion 1708 of the fastener 1704. Thereafter, paternity portion 1714 can be moved laterally 1724 along a longitudinal axis of the dressing portion according to the needs and circumstances of a particular patient. Once a tourniquet portion 1714 is properly positioned, the loop portion 1710 of the hook and loop fastener 1704 can be re-coupled 1726 to the hook portion 1708 of the fastener, thereby securing the tourniquet portion 1714 in place. Thereafter, according to one embodiment of the invention, handle 1716 can be used to twist, and thereby tighten the tourniquet portion 1714.

In certain embodiments of the invention, as shown in FIG. 17C, the tourniquet portion 1714 includes a loop portion 1728. The loop portion 1728 defines a cavity 1730 adapted to receive the handle 1716 therethrough, so as to facilitate twisting of the tourniquet.

Referring again to FIG. 17A one sees that rotation of the handle 1716 results in a twisted portion 1732 of the tourniquet portion 1714. According to certain embodiments, the invention includes a method of disposing a region of the twisted portion 1732 and/or a portion of the handle 1716 between the hook portion 1708 and the loop portion 1710 of the fastener 1704 so as to retain the tourniquet portion 1714 in a constricted configuration about a wounded limb of a patient. It should be appreciated that in various methods according to the invention, it is possible to subsequently release the loop portion 1710 of the fastener 1704 from the hook portion 1708 of the fastener to allow further adjustment of a circumferential tension and/or longitudinal position of the tourniquet portion 1714.

In another embodiment of the invention, the tourniquet portion includes a detent device adapted to readily include one way constrictive motion of the tourniquet strap. A first end of the tourniquet strap is coupled to be detent device and a second end of the tourniquet strap is passed through the detent device. Pulling on the second end of the strap tightens the tourniquet about, for example, a limb. In one embodiment, a release device on the detent device allows the tourniquet strap to be subsequently loosened.

FIG. 18A shows an exemplary wound dressing 1800 according to a further embodiment of the invention. In the illustrated embodiment, the wound dressing includes a first portion 1802 adapted to be disposed adjacent to a wound. The illustrated first portion 1802 is generally comparable to the truncated limb dressing 1700 of FIG. 17. When a skill in the art will appreciate, however, that the first portion 1802 could just as well be a dressing of the style presented as dressing 100 in FIG. 1, or any of a wide variety of other appropriate dressing configurations. Coupled to the first portion 1802 is a wrapping portion 1804. In the illustrated embodiment, the wrapping portion 1804 includes a substantially elastic textile material including, for example, a plurality of silverized deep groove nylon fibers. In various embodiments, the wrapping portion 1804 also includes a fiber batting material including a one or more of analgesic and hemostatic materials.

In the illustrated embodiment, the wrapping portion 1804 includes a plurality of capture devices 1806. FIG. 18B shows an exemplary capture device 1806 in additional detail. The capture device 1806 includes a grommet portion 1808 and a hook portion 1810. Grommet portion 1808 includes first 1812 and second 1814 flange portions and a tubular portion 1816 disposed between the first 1812 and second 1814 flange portions. The grommet portion 1808 is adapted to be coupled to the wrapping portion 1804 in a matter typically employed, and well understood in the art of grommets and riveting. Grommet portion 1808 includes an inner surface 1809 defining a bore therethrough. In various embodiments, the capture device 1806 includes a brass material, stainless steel material, an aluminum material another metallic material, a polymer material including, for example polyamide, polyamide, polyethylene (including, for example, ultrahigh molecular weight polyethylene) or any other appropriate material available and known in the art at the time of manufacture.

The dressing 1800 is applied by positioning first portion 1802 adjacent to wounded flesh. Thereafter, the wrapping portion 1804 is disposed about an additional portion of a patient's body, either in a helical wrapping arrangement or otherwise, as appropriate to particular clinical circumstances. When an appropriate degree of wrapping has been completed, a hook portion 1810 of one capture device 1806 is disposed within a bore of another capture device to secure the wrapping in place. Thereafter, any excess portion of the wrapping can be severed from the dressing and employed elsewhere in treatment of the same or another patient. One of skill in the art will appreciate that alternative fastening means, including, for example, a hook and loop (Velcro®) fastener is included in alternative embodiments of the invention.

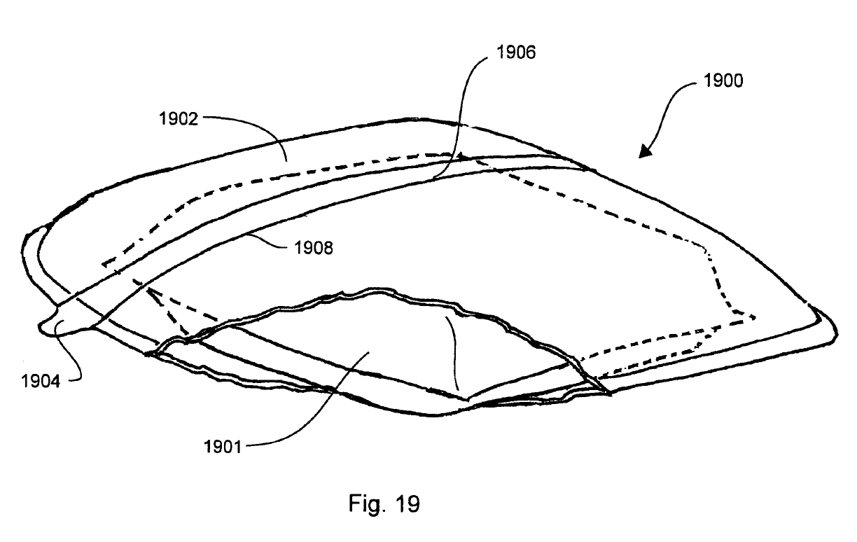

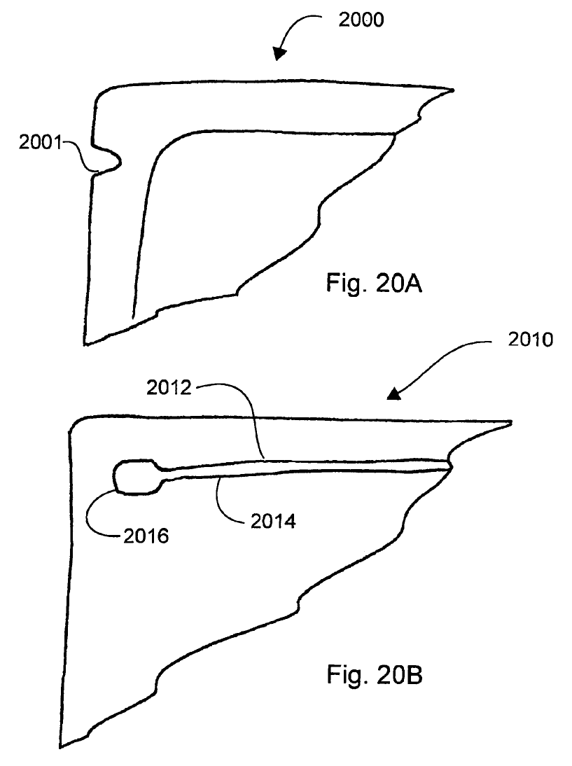

FIG. 19 shows, in cutaway perspective view, a wound dressing package 1900 according to one embodiment of the invention. The wound dressing package 1900 includes a wound dressing 1901 and a substantially gas and liquid impermeable envelope 1902. In certain embodiments, the package is adapted to sustain a substantial vacuum or an atmosphere of substantially nonreactive liquid or gas, therewithin. In various embodiments, the envelope includes a synthetic polymer material such as, for example, (Mylar), polyamide, polyaramid, polyethylene or other polymer material such as would be known to the creative practitioner of ordinary skill in the art. In a further embodiment of the invention, the envelope includes a reinforcing material such as, for example, reinforcing fibers. Depending on the particular application, the reinforcing fibers may include polymer fibers, glass fibers, carbon fibers, or other fibers or reinforcing devices appropriate for reinforcing a polymer material, as would be known to the creative practitioner of ordinary skill in the art.

As will be described below in additional detail, the envelope 1902 is adapted to be robust against inadvertent opening, but readily openable when required. Thus, for example, in the embodiment illustrated in FIG. 19, the envelope 1902 includes a pull-tab 1904. The pull-tab 1904 is coupled to, or integrally formed with, a pull-strip portion of the envelope 1906 disposed adjacent to a region of the envelope 1908 that is adapted to tear, or otherwise separate, when an appropriate force is applied between the pull-tab 1904 and the balance of the envelope 1902.

In the illustrated embodiment, the envelope contains a single item wound dressing 1901. According to one embodiment, the wound dressing 1901 is substantially as shown and described in relation to FIG. 1.

FIG. 20A shows a portion of a further embodiment of the invention including a wound dressing package 2000 having a notch 2001 at an edge thereof. The notch 2001 is adapted to facilitate opening of the wound dressing package 2000 by tearing.

FIG. 20B shows a portion of another embodiment of the invention including a wound dressing package 2010. In the illustrated embodiment, the wound dressing package 2010 includes a zipper device 2012 having a coupling portion 2014 and a tab portion 2016. The tab portion is drawn along a longitudinal axis of the coupling portion to release a wound dressing disposed within the package 2010. In another embodiment, the zipper device is replaced by a ripcord device.

In certain embodiments, the invention includes applying a dressing as described above in combination with modulated pulse wave ultrasonics. In other embodiments, a wound surface is pre-saturated with sterile water. In still other embodiments, vacuum pulse or hyperbaric conditions are applied in conjunction with a dressing as described above. In still further embodiment of the invention, an electric field is applied in conjunction with a dressing as described above.

While the exemplary embodiments described above have been chosen primarily from the field of emergency trauma wound dressings, one of skill in the art will appreciate that the principles of the invention are equally well applied, and that the benefits of the present invention are equally well realized in a wide variety of other dressing systems including, for example, postsurgical dressing systems. Further, while the invention has been described in detail in connection with the presently preferred embodiments, it should be readily understood that the invention is not limited to such disclosed embodiments. Rather, the invention can be modified to incorporate any number of variations, alterations, substitutions, or equivalent arrangements not heretofore described, but which are commensurate with the spirit and scope of the invention. Accordingly, the invention is not to be seen as limited by the foregoing description, but is only limited by the scope of the appended claims.

1. A wound dressing comprising:
a physical stanching member;
an integrated chemical coagulant material; and
an biocidal material.

2. A wound dressing as defined in claim 1 wherein said physical stanching member comprises a substantially porous jacket and absorptive bat.

3. A wound dressing as defined in claim 2 wherein said substantially porous jacket comprises a knitted textile material.

4. A wound dressing as defined in claim 2 wherein said substantially porous jacket comprises a woven textile material;

5. A wound dressing as defined in claim 2 wherein said substantially porous jacket comprises a nonwoven textile material;

6. A wound dressing as defined in claim 2 wherein said substantially porous jacket comprises a perforated sheet material;

7. A wound dressing as defined in claim 2 wherein said substantially porous jacket comprises a natural polymer material.

8. A wound dressing as defined in claim 2 wherein said substantially porous jacket comprises synthetic polymer material.

9. A wound dressing as defined in claim 8 wherein said synthetic polymer material comprises a polyamide material.

10. A wound dressing as defined in claim 8 wherein said synthetic polymer material comprises a polyaramid material.

11. A wound dressing as defined in claim 8 wherein said synthetic polymer material comprises a polyethylene material.

12. A wound dressing as defined in claim 8 wherein said synthetic polymer material comprises a polypropylene material.

13. A wound dressing as defined in claim 8 wherein said synthetic polymer material comprises a polybutylene material.

14. A wound dressing as defined in claim 2 wherein said absorptive bat comprises: a felted fiber material.

15. A wound dressing comprising:
a porous sheath;
an absorbent cushion, said absorbent cushion including an biocidal material; and
a coagulant material, said coagulant material being dispersed within said absorbent cushion.

16. A wound dressing comprising:
a substantially porous sheath, said porous sheath including a textile material;
a first fibrous layer, said first fibrous layer including a coagulant material; and
a second fibrous layer, said second fibrous layer including an inorganic biocidal agent.

17. A wound dressing as defined in claim 15 wherein said coagulant material comprises a hemostatic chemical material.

18. A wound dressing as defined in claim 17 wherein said hemostatic chemical material comprises a granulated Chitosin material.

19. A wound dressing as defined in claim 15 wherein said inorganic biocidal agent comprises silver.

20. A wound dressing as defined in claim 19 wherein said silver comprises a layer of 99.99% pure silver.

21. A wound dressing as defined in claim 15 wherein said textile material comprises a woven textile material.

22. A wound dressing as defined in claim 15 wherein said textile material comprises a knitted textile material.

23. A wound dressing as defined in claim 15 wherein said textile material comprises a non-woven textile material.

24. A wound dressing as defined in claim 15 wherein said textile material comprises a felt material.

25. A wound dressing as defined in claim 15 wherein said textile material comprises a natural polymer material.

26. A wound dressing as defined in claim 15 wherein said textile material comprises a synthetic polymer material.

27. A wound dressing as defined in claim 26 wherein said synthetic polymer material comprises a polyamide material.

28. A wound dressing as defined in claim 26 wherein said synthetic polymer material comprises a polyaramid material.

29. A wound dressing as defined in claim 26 wherein said synthetic polymer material comprises a polyethylene material.

30. A wound dressing as defined in claim 29 wherein said polyethylene material comprises high density polyethylene.

30. A wound dressing as defined in claim 29 wherein said polyethylene material comprises low-density polyethylene.

31. A wound dressing as defined in claim 15 wherein said textile material comprises a polymer fiber.

32. A wound dressing as defined in claim 31 wherein said polymer fiber comprises a longitudinal fiber having a polymer inner portion and a metallic outer portion.

33. A wound dressing as defined in claim 32 wherein said metallic outer portion comprises a metallic layer, said metallic layer including a silver material.

34. A wound dressing as defined in claim 33 wherein said metallic layer comprises a layer of silver non-electrolytically coupled to said polymer inner portion by application of ultrasonic energy.

35. A wound dressing as defined in claim 15 wherein said wound dressing further comprises a substantially nonporous package disposed outwardly of the said substantially porous sheath.
